(19) United States
(12) Patent Application Publication
(10) Pub. No.: US 2019/0314607 A1
(43) Pub. Date: Oct. 17, 2019

(54) CATHETER SYSTEM FOR ABLATION

(71) Applicants: Mehdi Razavi, Houston, TX (US); Mathews John, Houston, TX (US); Anand Ganapathy, Los Angeles, CA (US); Manuel Andres Gutierrez, Houston, TX (US); Vincent Ngo, Houston, TX (US); Loren Chane Sladek, Houston, TX (US); Brian Greet, Houston, TX (US); David Burkland, Houston, TX (US)

(72) Inventors: Mehdi Razavi, Houston, TX (US); Mathews John, Houston, TX (US); Anand Ganapathy, Los Angeles, CA (US); Manuel Andres Gutierrez, Houston, TX (US); Vincent Ngo, Houston, TX (US); Loren Chane Sladek, Houston, TX (US); Brian Greet, Houston, TX (US); David Burkland, Houston, TX (US)

(21) Appl. No.: 16/381,886

(22) Filed: Apr. 11, 2019

Related U.S. Application Data
(60) Provisional application No. 62/657,191, filed on Apr. 13, 2018.

(51) Int. Cl. A61M 25/00; A61B 18/14; A61M 25/10

(52) U.S. Cl. CPC A61M 25/0084; A61B 18/1492; A61B 2018/00029; A61M 25/1002; A61M 2025/0096; A61B 18/1477

(57) ABSTRACT

A catheter system includes a head including ports, a tail including conduits, and a medical balloon located between the head and the tail. Each conduit is connected to one of the ports. The medical balloon comprises ridges located around a circumference of the medical balloon. One or more sensors are located in the conduits. The catheter system can be used for ablation of the left atrial appendage using an ablation agent. The catheter system is capable of sealing the left atrial appendage, administering the ablation agent, delivering rinsing fluid into the sealed cavity, and extracting liquid from the sealed cavity.

### CROSS-REFERENCE TO RELATED APPLICATIONS

This application claims priority to U.S. provisional application Ser. No. 62/657,191 filed on Apr. 13, 2018, the content of which is included herein by reference.

### BACKGROUND

This disclosure relates generally to apparatus and methods for ablation of the left atrial appendage or other tissue using, for example, alcohol as the ablation agent. More particularly, this disclosure relates to a catheter system capable of sealing the left atrial appendage or a similar cavity, administering alcohol or another ablation agent, delivering saline or other rinsing fluids into the sealed cavity, and extracting liquid from the sealed cavity.

U.S. App. Pub. No. 2008/0045890 discloses methods and systems that rely on the injection of ethanol and other tissue-ablating agents for ablating tissue.

U.S. App. Pub. No. 2014/0121641 discloses an intravascular catheter for peri-vascular and/or peri-urethral tissue ablation that includes multiple needles advanced through guide tubes which may be supported by an expandable balloon.

U.S. App. Pub. No. 2006/0190022 discloses a method for injecting a therapeutic agent into a target tissue.

Despite these advances, there is a continuing need in the art for methods and apparatus for ablation of the left atrial appendage using an ablation agent.

### BRIEF SUMMARY OF THE DISCLOSURE

In one aspect, a catheter system comprises a head including a plurality of ports, a tail including a plurality of conduits, each of the plurality of conduits being connected to one of the plurality of ports, and a medical balloon located between the head and the tail. The medical balloon comprises at least one ridge located around a circumference of the medical balloon.

The at least one ridge may be made of flexible plastic, including but not limited to latex.

The at least one ridge may be formed by a hollow tube.

The at least one ridge may be designed to extend radially upon inflation of the medical balloon.

The at least one ridge may be designed to elongate the inflated shape of the medical balloon.

In another aspect, a catheter system comprises a head including a plurality of ports, a tail including a plurality of conduits, each of the plurality of conduits being connected to one of the plurality of ports, and a medical balloon located between the head and the tail. An alcohol sensor is located in at least one of the plurality of conduits.

The alcohol sensor may include an electrolytic sensor.

The alcohol sensor may comprise an anode disposed in the catheter system near one of the plurality of ports, a cathode, and an electric current measuring device connected between the anode and the cathode.

### BRIEF DESCRIPTION OF THE DRAWINGS

For a more detailed description of the embodiments of the disclosure, reference will now be made to the accompanying drawings, wherein:

FIG. 1 is a sectional view of a catheter system in accordance with an embodiment, wherein the catheter system is illustrated sealing the left atrial appendage;

FIG. 2 is a sectional view of a distal end of the catheter system shown in FIG. 1;

FIG. 3 is a side view of the distal end of the catheter system shown in FIG. 1;

FIG. 4 is a view of a catheter system in accordance with an embodiment;

FIG. 5 is a perspective view of a distal end of the catheter system shown in FIG. 4;

FIG. 6 is a perspective view of the distal end of the catheter system shown in FIG. 4, wherein a balloon is illustrated in a deflated configuration;

FIG. 7 is a perspective view of the distal end of the catheter system shown in FIG. 4, wherein a balloon is illustrated in an inflated configuration; and

FIGS. 8A-8F are schematics illustrating a sequence of operation of a catheter system.

### DETAILED DESCRIPTION

It is to be understood that the following disclosure describes several exemplary embodiments for implementing different features, structures, or functions of the invention. Exemplary embodiments of components, arrangements, and configurations are described below to simplify the disclosure; however, these exemplary embodiments are provided merely as examples and are not intended to limit the scope of the invention. Additionally, the disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the various Figures. Finally, the exemplary embodiments presented below may be combined in any combination of ways, i.e., any element from one exemplary embodiment may be used in any other exemplary embodiment, without departing from the scope of the disclosure.

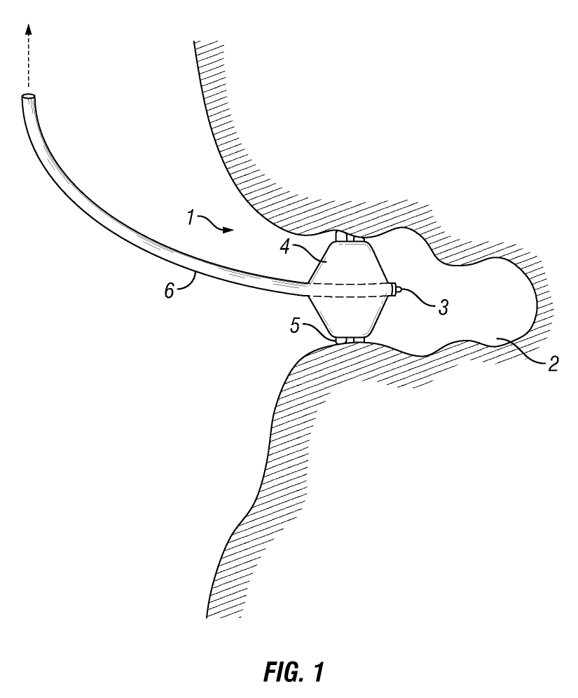

Referring initially to FIG. 1, a catheter system is illustrated in the heart of a patient. The catheter system is shown inserted in the left atrium 1 of the heart, extending into an interior of the left atrium appendage 2.

The catheter system comprises a head 3. The head 3 provides ports connected to conduits, such as transport lumens disposed inside a catheter tail 6. As such, fluids can be administered to and extracted from the left atrium appendage 2. Catheter tail 6 is partially shown and normally extends further to exit the body of the patient.

A distal end of the catheter system comprises a medical balloon 4, which is located between the head 3 and the catheter tail 6. The medical balloon 4 provides a means for sealing off the left atrium appendage 2 from the left atrium 1 of the heart. The medical balloon 4 is provided with one or more ridges 5. Ridges 5 may be made of flexible plastic, such as latex, and may be hollow, such as a hollow tube having its two ends connected. Materials other than latex may be used as flexible plastic. Ridges 5 may be located around an entire circumference of the medical balloon 4. Ridges 5 are extended radially upon inflation of the medical balloon 4 and provide surfaces capable of sealing against the wall of the left atrium appendage 2 despite the complex geometry of the left atrium appendage 2. Ridges 5 control (for example, elongate) the inflated shape of the medical balloon 4. As such, the medical balloon 4 provided with ridges 5 can generally provide better sealing of the left atrium appendage 2 from the left atrium 1 of the heart than the sealing provided by the medical balloon 4 alone.

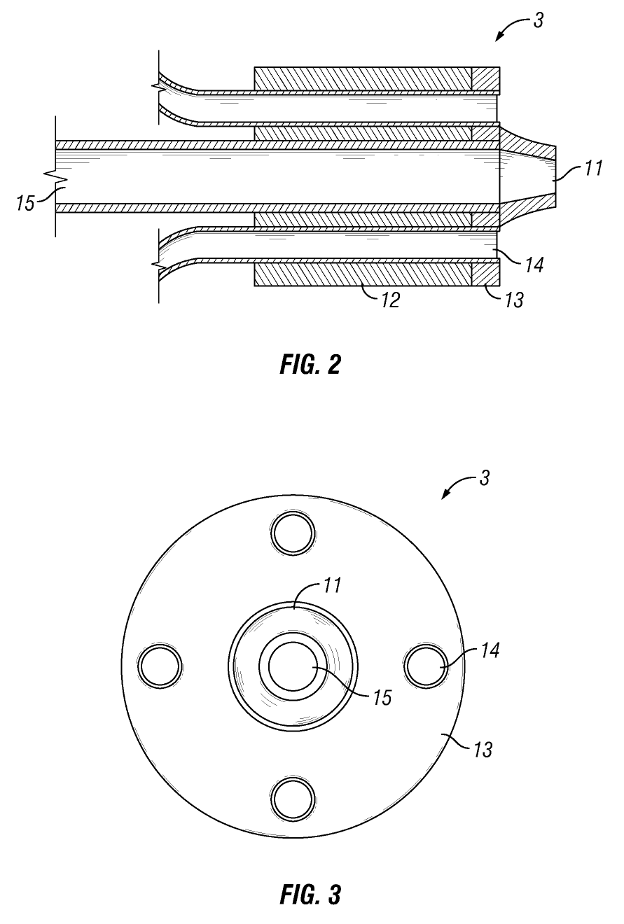

Turning to FIGS. 2 and 3, the distal end of the catheter system comprises a headshell 12 and a head cap 13. The headshell 12 may be formed as a cylinder and made of rigid plastic or biologically inert metal. The headshell 12 is used to guide several lumens (e.g., five lumens, including main lumen 15 and four auxiliary lumens 14) that run through the catheter tail 6 toward the head cap 13. The head cap 13 may be formed as a circular disc or as another shape, and may also be made of rigid plastic or biologically inert metal. The head cap 13 is perforated to provide holes (e.g., five holes). The holes may be sized so that the ends of the lumens fit into the holes. The head cap 13 may be designed to be fastened on the headshell 12 by pressing.

The lumens that run through the catheter tail 6 toward the head cap 13 can include, for example, the main lumen 15 and four auxiliary lumens 14. The lumens may consist of flexible thin-walled tubing, which may be made of silicone polymer. The main lumen 15 may have a larger cross-section than the auxiliary lumens 14. The main lumen 15 is preferably used for administering alcohol or another ablation agent, and/or optionally for extracting fluid after ablation. Some of the auxiliary lumens 14 (e.g., two diametrically opposed auxiliary lumens) are preferably used for delivering rinsing fluids to the left atrium appendage 2 (in FIG. 1), while other of the auxiliary lumens 14 (e.g., two other diametrically opposed auxiliary lumens) are preferably used for extracting fluids from the left atrium appendage 2. The rinsing fluids typically include saline and/or blood.

The hole that receives the main lumen 15 may occupy a central location of the head cap 13 and may be evenly surrounded by the holes that receive the auxiliary lumens 14. The central port of the head cap 13 is fitted with a nozzle 11. The nozzle 11 is preferably designed for having an inlet-outlet area ratio that disturbs the laminar flow in the main lumen 15 and generates a transition flow regime. As such, the fluid exiting the nozzle 11 spreads laterally. Optionally, the head cap 13 may also comprise several lateral holes 20 (as shown in FIG. 5, e.g., four peripheral ports) in communications with some or all of the four auxiliary lumens 14.

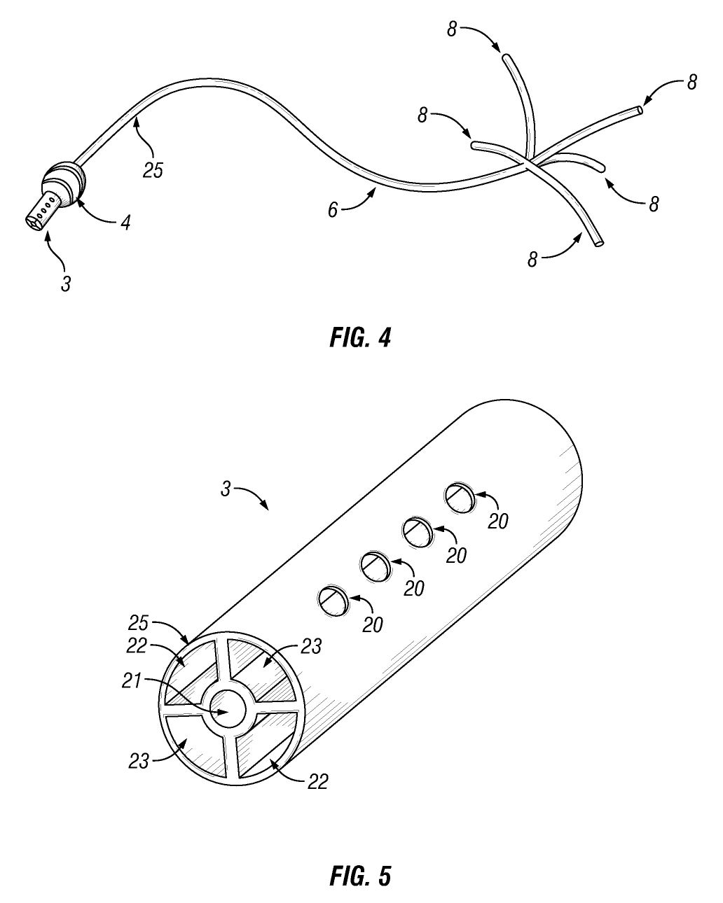

Turning to FIG. 4, an alternative embodiment of a catheter system is illustrated. Similarly to the catheter system shown in FIGS. 1-3, the catheter system includes a catheter tail 6, a head 3 located at a distal end of the catheter, and a medical balloon 4 located between the head 3 and the catheter tail 6. However, all or a portion of the catheter tail 6 and/or the head 3 may be made of a single tube 25 that can, in some embodiments, be partitioned by walls into several sections (e.g., one central, circular section, and four peripheral, arc sections). Each section forms a conduit. As shown in FIG. 4, a proximal end of the catheter system may, in some embodiments, comprise separate tubes 8 corresponding to each of the sections.

Turning to FIG. 5, the head 3 provides ports connected to the sections of the single tube 25 making at least a portion of the catheter tail 6. The distal end of the central, circular section forms a central port 21, which may be used to administer alcohol or another ablation agent. The distal ends of two of the four peripheral, arc sections form two peripheral ports 23, which can be generally diametrically opposed, and which may be used to deliver rinsing fluids such as saline and/or blood. The distal ends of the other two of the four peripheral arc sections form two peripheral ports 22, which can be generally diametrically opposed, and which may be used to extract fluid. In additions, lateral holes 20 may be provided across the wall of the single tube 25, optionally communicating with one of the four peripheral sections, to better distribute the flow of rinsing fluid delivered to the left atrium appendage 2 and/or better distribute the flow of fluid extracted from the left atrium appendage 2. While FIG. 5 shows four longitudinally distributed lateral holes communicating with one of the peripheral sections, any number of lateral holes may be provided. Also, lateral holes may be distributed circumferentially so that a subset of the lateral holes 20 may communicate with one of the four peripheral sections, and another subset of the lateral holes 20 may communicate with another one of the four peripheral sections. For example, each of the peripheral sections may be provided with one or more lateral holes 20.

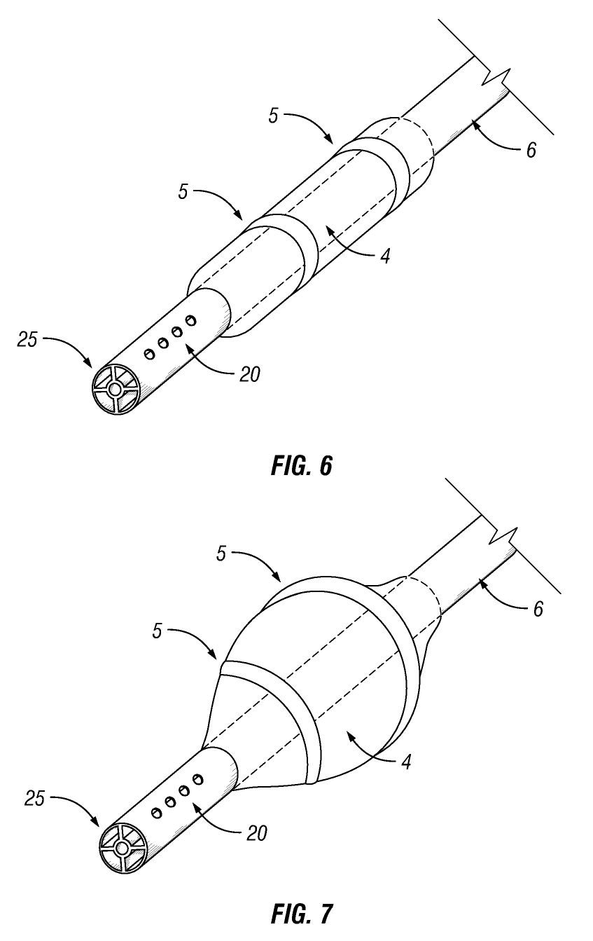

Turning to FIGS. 6 and 7, the medical balloon 4, including the ridges 5, is illustrated respectively in deflated and inflated configurations. The ridges 5 are similar to the ridges 5 illustrated in FIG. 1.

The catheter system shown in FIG. 1 and/or 4 may be equipped with one or more sensors at least partially located near the head 3. For example, an alcohol sensor may be implemented as an electrolytic sensor and may comprise an anode disposed in the catheter system near one of the ports used for extracting fluid from the left atrium appendage 2, and a cathode outside the body of the patient. The anode oxidizes alcohol (mixed with water from the rinsing solution) into acetic acid as alcohol moves past the anode, and the cathode reduces atmospheric oxygen and hydrogen into water. The electric current generated between the anode and the cathode is indicative of alcohol content of the fluid contacting the anode. In another example, a pressure sensor may be implemented with a strain gauge fixed on a body that deforms with pressure.

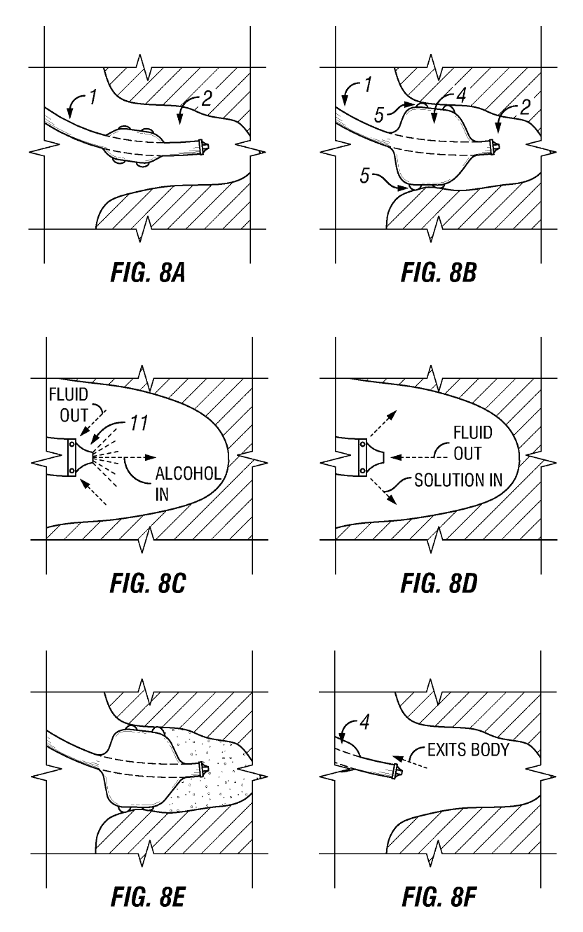

Turning to FIGS. 8A-8E, a sequence of steps of an ablation procedure of the left atrial appendage is illustrated. The procedure is to treat atrial fibrillation in a patient and to prepare the left atrial appendage for closure with another device such as the WATCHMAN implant provided by Boston Scientific. However, the catheter system disclosed herein may alternatively be used to seal off different areas of the body, such as veins, arteries, and other tubular organs, in order to administer drugs or possibly a blood thinning agent to these areas.

The ablation procedure begins with the entrance of the catheter system, such as described herein with respect to FIG. 1 or 4, into the left atrium of the heart. This entrance can be done through the radial or femoral artery to gain access to the aorta (and thus the left atrium) or by creating an incision in the heart tissue deep enough to allow the catheter system to be introduced to the left atrium immediately from outside the body of the patient. Once the distal end of the catheter system is inside the left atrium, the distal end is navigated to the left atrium appendage 2 and situated such that the balloon can expand and make contact with the appendage orifice. In FIG. 8A, the catheter system is illustrated inside the left atrium appendage 2 of the heart of a patient. In FIG. 8B, the medical balloon 4 is inflated to extend the ridges 5 toward the walls of the left atrium appendage 2 and seal off the left atrium appendage 2 from the left atrium 1. The medical balloon 4 can be expanded by injecting a sufficient volume of air in the balloon to deform the medical balloon 4 to create a good seal. A flush of contrast under fluoroscopy can confirm the seal. In FIG. 8C, alcohol or other ablation agent is administered through a central port 21 (e.g., through the nozzle 11), while fluid present in the sealed portion of the left atrium appendage 2 is extracted via two peripheral ports 22, and optionally via lateral holes 20 (better shown in FIG. 5). Preferably, the pressure in the sealed portion of the left atrium appendage 2 is maintained constant. For example, a pressure sensor provided at the distal end of the catheter system may be used to monitor pressure and control the flow rate of alcohol. The administration of alcohol is ceased when sufficient tissue is ablated. For example, once a known volume of alcohol has been administered, the main port may begin to extract the excess alcohol slowly. In FIG. 8D, saline, blood or other rinsing fluid is delivered via the other two peripheral ports 23, and optionally via lateral holes 20 while fluid and/or ablation debris present in the sealed portion of the left atrium appendage 2 are still extracted via the same two peripheral ports 22, and optionally via lateral holes 20, as shown for example in FIG. 8C. Again, the pressure in the sealed portion of the left atrium appendage 2 is preferably maintained constant, for example as previously discussed. In FIG. 8E, the rinsing is continued until little to no alcohol remains in the left atrium appendage 2. To ensure that little to no alcohol remains in the left atrium appendage 2, the alcohol content of the fluid extracted from the left atrium appendage 2 may be monitored using an alcohol sensor. In FIG. 8F, the medical balloon 4 is deflated and the catheter system is extracted from the patient body.

While the disclosure is susceptible to various modifications and alternative forms, specific embodiments thereof are shown by way of example in the drawings and description. It should be understood, however, that the drawings and detailed description are not intended to limit the invention to the particular form disclosed, but on the contrary, the intention is to cover all modifications, equivalents, and alternatives falling within the scope of the claims.

What is claimed is:

1. A catheter system, comprising:
a head including a plurality of ports;
a tail including a plurality of conduits, each of the plurality of conduits being connected to one of the plurality of ports; and
a medical balloon located between the head and the tail,
wherein the medical balloon comprises at least one ridge located around a circumference of the medical balloon.

2. The catheter system of claim 1, wherein the at least one ridge is made of flexible plastic.

3. The catheter system of claim 1, wherein the at least one ridge is formed by a hollow tube.

4. The catheter system of claim 1, wherein the at least one ridge is designed to extend radially upon inflation of the medical balloon.

5. The catheter system of claim 1, wherein the at least one ridge is designed to elongate an inflated shape of the medical balloon.

6. The catheter system of claim 1, further comprising an alcohol sensor, wherein the alcohol sensor is located in at least one of the plurality of conduits.

7. The catheter system of claim 6, wherein the alcohol sensor includes an electrolytic sensor.

8. The catheter system of claim 7, wherein the electrolytic sensor comprises:
an anode disposed in the catheter system near one of the plurality of ports;
a cathode; and
an electric current measuring device connected between the anode and the cathode.

9. A catheter system, comprising:
a head including a plurality of ports;
a tail including a plurality of conduits, each of the plurality of conduits being connected to one of the plurality of ports;
a medical balloon located between the head and the tail; and
an alcohol sensor,
wherein the alcohol sensor is located in at least one of the plurality of conduits.

10. The catheter system of claim 9, wherein the alcohol sensor includes an electrolytic sensor.

11. The catheter system of claim 10, wherein the electrolytic sensor comprises:
an anode disposed in the catheter system near one of the plurality of ports;
a cathode; and
an electric current measuring device connected between the anode and the cathode.

12. The catheter system of claim 11, wherein the medical balloon comprises at least one ridge located around a circumference of the medical balloon.

13. The catheter system of claim 12, wherein the at least one ridge is made of flexible plastic.

14. The catheter system of claim 13, wherein the at least one ridge is formed by a hollow tube.

15. The catheter system of claim 14, wherein the at least one ridge is designed to extend radially upon inflation of the medical balloon.

16. The catheter system of claim 15, wherein the at least one ridge is designed to elongate an inflated shape of the medical balloon.
